(19) United States
(12) Patent Application Publication
(10) Pub. No.: US 2024/0173279 A1
(43) Pub. Date: May 30, 2024

(54) Method of Treatment for Autophagy Diseases by Administration of Dexibuprofen and Use of Dexibuprofen for Preparation of a Medicament for Same

(71) Applicant: JEM THERAPEUTICS PBC, Delray Beach, FL (US)

(72) Inventors: Rebecca Kusko, Cambridge, MA (US); Renan Antonio Escalante Chong, Cambridge, MA (US); Rajaraman KRISHNAN, Cambridge, MA (US); Stefania Risso, Cambridge, MA (US); Jermaine Ross, Cambridge, MA (US); Yoonjeong Cha, Cambridge, MA (US); Benjamin Zeskind, Cambridge, MA (US)

(73) Assignee: JEM THERAPEUTICS PBC, Delray Beach, FL (US)

(21) Appl. No.: 18/493,414

(22) Filed: Oct. 24, 2023

Related U.S. Application Data
(63) Continuation of application No. PCT/US2022/026299, filed on Apr. 26, 2022.
(60) Provisional application No. 63/179,675, filed on Apr. 26, 2021.

(51) Int. Cl. A61K 31/192; A61P 21/00

(52) U.S. Cl. CPC A61K 31/192; A61P 21/00

(57) ABSTRACT

Some embodiments relate to a method of treating autophagy diseases in a patient in need thereof, comprising administering to the patient a composition comprising a therapeutically effective amount of dexibuprofen or a pharmaceutically acceptable salt thereof. In some embodiments, the method comprises treating diseases related to a TECPR2 mutation, including treatment of Hereditary Spastic Paraparesis in the patient.

### CROSS-REFERENCE TO RELATED APPLICATIONS

This is a continuing application, under 35 U.S.C. § 120, of copending international application No. PCT/US2022/026299, filed Apr. 26, 2022, which designated the United States and was published in English, which application claims the priority, under 35 U.S.C. § 119, of U.S. Provisional Patent Application Ser. No. 63/179,675, filed Apr. 26, 2021; the prior applications are herewith incorporated by reference in their entirety.

### STATEMENT REGARDING FEDERALLY SPONSORED RESEARCH OR DEVELOPMENT

Not Applicable

### FIELD OF THE INVENTION

The present disclosure relates to a method of treatment for autophagy diseases by administration of Dexibuprofen and use of Dexibuprofen for preparation of a medicament for same.

### BACKGROUND OF THE INVENTION

Connecting human disease and chemical perturbation genomic data offers an unprecedented opportunity to inform the development of new medicines. A critical barrier is the computational and intellectual challenge of creating methodologies that are 1) efficient and scalable to leverage these data, 2) capable of identifying true positives among the sea of noise in pre-clinical and human genomic data, and 3) able to integrate measurements in a biologically interpretable way to facilitate both mechanistic understanding and experimental follow-up.

Approaches like CMAP, probabilistic CMAP and XSum seek to link disease and perturbation genomic signatures. These approaches require a cumbersome step of splitting the genomic signatures into up and down-regulated gene lists and compute statistical significance through a computationally expensive approach. For the pharmaceutical and biotechnology industry, results from many of these tools are hard to take action on, due to challenges in interpretability. To address these challenges, we implemented an R package called “Cosiner”, our Disease Cancelling Technology (DCT). Details of the approach are described in the methods of our previous publication (Kusko, R. et al., Large-scale transcriptomic analysis reveals that pridopidine reverses aberrant gene expression and activates neuroprotective pathways in the YAC128 HD mouse. Mol. Neurodegener. 13, 25 (2018)).

TECPR2 disease is a rare and autosomal recessive disease (OMIM 615031). The TECPR2 protein is multi-functional and responsible for autophagosome formation, scaffolding, protein trafficking, nuclear RNA trafficking (Stadel, D. et al., TECPR2 Cooperates with LC3C to Regulate COPII-Dependent ER Export. Mol. Cell 60, 89-104 (2015)). TECPR2 is activated by ER stress. Many different mutations of the TECPR2 gene lead to disease states in humans. A 1 bp frameshift deletion in the TECPR2 gene leads to a premature stop codon, causing degradation of the TECPR2 protein (Oz-Levi, D. et al., Mutation in TECPR2 reveals a role for autophagy in Hereditary Spastic Paraparesis (HSP). Am. J. Hum. Genet. 91, 1065-1072 (2012)). Other mutations lead to single amino acid changes within the TECPR2 protein. Heterozygote carriers of theses TECPR2 mutations do not appear to have symptoms. However, homozygote children with two mutant copies of the TECPR2 gene are afflicted with TECPR2 related diseases, such as Hereditary Spastic Paraparesis, a group of neurodegenerative disorders that affect about 10 out of 100,000 individuals and characterized by corpus collosum thinning, intellectual disability, central hypoventilation, GI reflux, wake apnea, areflexia, and dysmorphia. See id. In some instances, homozygosity for mutant TECPR2 genes can be lethal in childhood.

### SUMMARY OF THE INVENTION

Some embodiments of the present invention relate to a method of treating autophagy diseases in a patient in need thereof, comprising administering to the patient a composition comprising a therapeutically effective amount of Dexibuprofen or a pharmaceutically acceptable salt thereof.

In some embodiments, the method comprises treating diseases related to a TECPR2 mutation, including Hereditary Spastic Paraparesis in the patient. In one embodiment, Dexibuprofen has the following formula:

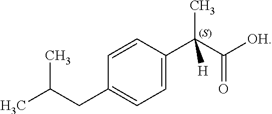

In another aspect, the composition further comprises a pharmaceutically acceptable carrier.

In another aspect, the method further comprises administering another therapeutic agent to the patient.

In another aspect, the composition and the other therapeutic agent are co-administered.

In another aspect, both the composition and the other therapeutic agent are dispersed or dissolved together in a pharmaceutically-acceptable carrier.

In another aspect, the administration is an oral administration.

In another aspect, the oral administration is by a tablet comprising the composition.

In another aspect, the administration is intravenous or subcutaneous administration.

In another aspect, the administration is of a solution comprising Dexibuprofen or pharmaceutically acceptable salt in a concentration between 1 mg/ml and 500 mg/ml.

In another aspect, Dexibuprofen or pharmaceutically acceptable salt thereof administered to the patient is in an amount from about 0.1 mg to about 1,000 mg of Dexibuprofen per kg of body weight of the patient.

Other embodiments of the present invention relate to the use of Dexibuprofen or a pharmaceutically acceptable salt thereof in the preparation of a medicament for the treatment of autophagy diseases in a patient in need thereof. In some embodiments, the medicament is intended for treating diseases related to a TECPR2 mutation, including Hereditary Spastic Paraparesis in the patient.

In another aspect, the Autophagy disease is an Autophagy disease related to a TECPR2 mutation in the patient.

In another aspect, the method comprises treating Hereditary Spastic Paraparesis in the patient.

In another aspect, the medicament further comprises a pharmaceutically acceptable carrier.

In another aspect, the medicament further comprises another therapeutic agent.

In another aspect, the medicament is intended for oral administration, such as a tablet.

In another aspect, the medicament is intended for intravenous or subcutaneous administration.

In another aspect, the medicament comprises a solution containing Dexibuprofen or pharmaceutically acceptable salt in a concentration between 1 mg/ml and 500 mg/ml.

In another aspect, the medicament contains Dexibuprofen or pharmaceutically acceptable salt thereof in an amount from about 0.1 mg to about 1,000 mg of Dexibuprofen per kg of body weight of the patient.

In some embodiments, the use comprises the use of Dexibuprofen or a pharmaceutically acceptable salt thereof substantially as described in the specification.

These and other features, aspects, and advantages of the present invention will become better understood with reference to the following description and appended claims.

### BRIEF DESCRIPTION OF THE DRAWINGS

FIG. 1 is a series of plots showing genes responsible for the Dexibuprofen high disease cancelling score for TECPR2. Each dot represents a gene. Changes in gene expression caused by Dexibuprofen (y-axis) are plotted as a function of gene expression changes associated with each genetic perturbation represented (x-axis).

FIG. 2 is a dot plot showing a comparison of expression of three key genes between ibuprofen and Dexibuprofen. Drug induced gene expression level change is on the y-axis by the t-statistic, and gene expression level change in disease is on the x-axis as t-statistic. These three genes are regulated in a different direction between gene expression signatures of TECPR2 disease and Dexibuprofen (orange dots), and but are merely trending with no statistically significant p-value for ibuprofen (blue dots).

### DETAILED DESCRIPTION OF THE EMBODIMENTS

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Some embodiments described herein relate to a method of treating a patient with an autophagy disease by administration of Dexibuprofen. Autophagy diseases include, but are not limited to, Hereditary Spastic Paraparesis, Multiple Sclerosis, Huntington's disease, spinocerebellar ataxia, amyotrophic lateral sclerosis (ALS), Parkinson's disease, Alzheimer' s disease, Batten Disease, lewy body dementia, Vici syndrome, neuronal ceroid lipofuscinosis, pallidoluysian atrophy, spinobulbar muscular atrophy, cdkl5 disease, Charcot-Marie-Tooth disease, hereditary spastic paraplegia, Lafora disease, β-propeller protein-associated neurodegeneration (BPAN). Some embodiments described herein relate to a method of treating Autophagy diseases related to a TECPR2 mutation in a patient, particularly for a patient who is homozygous for mutations of the TECPR2 gene. In one embodiment, the method includes administering to the patient a composition comprising a therapeutically effective amount of dexibuprofen or a pharmaceutically acceptable salt thereof. The chemical name of dexibuprofen is (S)-a-methyl-4-(2-methylpropyl)benzeneacetic acid or S(+)-ibuprofen, and it has the following formula:

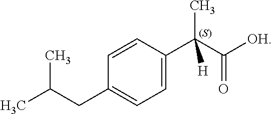

As used herein, a “therapeutically effective amount” refers to a sufficient amount of dexibuprofen to treat autophagy diseases in a patient, at a reasonable benefit/risk ratio applicable to any medical treatment. It will be understood, however, that the total daily usage of dexibuprofen will be decided by the attending physician within the scope of sound medical judgment. The specific therapeutically effective dose level for any particular subject will depend upon a variety of factors including the disorder being treated and the severity of the disorder; the specific composition employed, the age, body weight, general health, sex and diet of the patient; the time of administration, route of administration, and rate of excretion of dexibuprofen employed; the duration of the treatment; drugs used in combination or coincidental with dexibuprofen; and like factors well known in the medical arts. For example, it is well known within the skill of the art to start doses of the compound at levels lower than those required to achieve the desired therapeutic effect and to gradually increase the dosage until the desired effect is achieved. In addition, a “therapeutically effective amount” is the amount that will elicit the biological or medical response of a tissue, system, or subject that is being sought by a researcher or clinician.

One of skill in the art recognizes that an amount may be considered therapeutically “effective” even if the condition is not totally eradicated or prevented, but it or its symptoms and/or effects are improved or alleviated partially in the patient. Various indicators for determining the effectiveness of a method for treating diseases related to a TECPR2 mutation in a patient are known to those skilled in the art. Examples of suitable indicators include, but are not limited to, a reduction of morbidity or mortality in clinical outcomes, and/or other indicator of disease response.

In some embodiments, the composition comprises from about 5% to about 10%, 20%, 30%, 40%, 50%, 60%, 70%, 80%, 90%, or 95% by weight of dexibuprofen compound, and preferably from about 30% to about 90% by weight of dexibuprofen compound, based upon the total weight of the composition taken as 100% by weight.

Other ingredients may be included in the composition, such as other active agents, preservatives, buffering agents, salts, a pharmaceutically acceptable carrier, or other pharmaceutically-acceptable ingredients.

In some embodiments, the method further comprises administering another therapeutic agent to the patient. In some embodiments, the composition and the other therapeutic agent are co-administered. In one embodiment, the order of administering the composition and the other therapeutic agents can be in any order.

A potential advantage of utilizing dexibuprofen, or a pharmaceutically acceptable salt thereof, in combination with one or more additional agent(s) is that the use of two or more compounds having different mechanism of actions may bring better results for treating the disease compared to the result when a compound is administered as monotherapy.

In some embodiments, both the composition and the other therapeutic agent are dispersed or dissolved together in a pharmaceutically-acceptable carrier.

In some embodiments, the administration of the composition is of a solution comprising dexibuprofen or pharmaceutically acceptable salt in a concentration between 1 mg/ml and 500 mg/ml.

The dosage may range broadly, depending upon the desired effects and the therapeutic indication. The daily dosage regimen for an adult human patient may be, for example, an oral dose of between 0.01 mg and 3000 mg of dexibuprofen, preferably between 1 mg and 700 mg, e.g. 5 to 200 mg, or between about 0.1 mg and about 1,000 mg of dexibuprofen per kg of body weight of the patient. Exemplary doses for a medicament for treating autophagy diseases include 1 mg, 5 mg, 10 mg, 20 mg, 25, mg, 30 mg, 50 mg, 60 mg, 75 mg, 100 mg, 150 mg, 200 mg, 250 mg, 300 mg, 350 mg, and 500 mg per unit dosage form. The dosage may be a single one or a series of two or more given in the course of one or more days, as is needed by the patient. In some embodiments, dexibuprofen, or a pharmaceutically acceptable salt thereof, are administered for a period of continuous therapy, for example for a week or more, or for months or years. In some embodiments, dexibuprofen, or a pharmaceutically acceptable salt thereof, can be administered less frequently compared to the frequency of administration of an agent within the standard of care. In some embodiments, dexibuprofen, or a pharmaceutically acceptable salt thereof, can be administered one time per day. For example, dexibuprofen, or a pharmaceutically acceptable salt thereof, can be administered one time per day to a patient suffering from an Autophagy related disease, including those related to a TECPR2 mutation. In some embodiments, the total time of the treatment regime with dexibuprofen, or a pharmaceutically acceptable salt thereof, can be less compared to the total time of the treatment regime with the standard of care.

In instances where human dosages for dexibuprofen have been established for at least some condition, those same dosages may be used, or dosages that are between about 0.1% and 500%, more preferably between about 25% and 250% of the established human dosage.

In cases of administration of a pharmaceutically acceptable salt, dosages may be calculated as the free base. As will be understood by those of skill in the art, in certain situations it may be necessary to administer the composition disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety that are sufficient to maintain the modulating effects, or minimal effective concentration (MEC). Dosages necessary to achieve the MEC will depend on individual characteristics and route of administration. However, HPLC assays or bioassays can be used to determine plasma concentrations. Dosage intervals can also be determined using MEC value. Compositions should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Dexibuprofen is used for pain relief in Greece, Italy, Poland, Portugal, Romania, South Korea, and Spain. It crosses the blood brain barrier (BBB) like ibuprofen. Dexibuprofen is approved for use in children in some countries. Korea has published multiple studies in a pediatric population comparing dexibuprofen to ibuprofen and did not note safety differences (Kim et al., Pediatrics International 2013; Yoon et al., British Journal of Pharmacology 2008). Dexibuprofen's side effect profile is extremely similar to ibuprofen (Kaehler Inflammatory Pharmacology et al 2003).

The disclosed embodiments are suitable for various routes of administration, depending upon the particular carrier and other ingredients used. For example, the prophylactic and/or therapeutic compounds or compositions can be injected intramuscularly, subcutaneously, intradermally, or intravenously. In some embodiments, the administration is of a solution comprising dexibuprofen or pharmaceutically acceptable salt in a concentration between 1 mg/ml and 500 mg/ml. The composition can also be administered via mucosa, such as intranasally or orally (e.g., buccal or sublingual). In some embodiments, the oral administration is by a tablet comprising the composition. The compounds or compositions can also be administered through the skin via a transdermal patch.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine and lysine.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Compositions

Some embodiments described herein relates to a composition, which can include an effective amount of dexibuprofen, or a pharmaceutically acceptable salt thereof and a pharmaceutically acceptable carrier, diluent, excipient or combination thereof.

The term “composition” refers to a mixture of dexibuprofen with other chemical components, such as diluents or carriers. The composition facilitates administration of the compound to an organism. Compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Compositions will generally be tailored to the specific intended route of administration. A composition is suitable for human applications.

The term “medicament” refers to a composition of dexibuprofen or pharmaceutically acceptable salt thereof intended for use in the treatment of a disease or condition in a patient in need thereof. A medicament will generally contain a therapeutically effective amount of dexibuprofen, or a predetermined fraction thereof (such as half), and may also include other chemical components, such as diluents or carriers.

As used herein, a “carrier” refers to a compound that facilitates the incorporation of a compound into cells or tissues. For example, without limitation, dimethyl sulfoxide (DMSO), ethanol (EtOH), or PEG400 is a commonly utilized carrier that facilitates the uptake of many organic compounds into cells or tissues of a subject. Other carrier or vehicle is selected from fatty acids, fatty acid esters, fatty alcohols, fatty alcohol ethers, fatty alcohol esters, C2-C8 linear or branched, saturated or unsaturated alcohols, polyols, aromatic alcohols, alkyleneglycol ethers, alkyleneglycol esters and natural or mixtures thereof.

Suitable fatty alcohols according to the present invention are C6-C24 alcohols from vegetable and animal fats and oils, such as 2-octyldodecanol, 2-ethylhexanoyl alcohol, arachidyl alcohol, behenyl alcohol, caprylic alcohol, caproyl alcohol, capric alcohol, castor oil alcohol, ceterayl alcohol, palmityl (cetyl) alcohol, coconut alcohol, cotton alcohol, decyl alcohol, elaidyl alcohol, erucyl alcohol, gadoleyl alcohol, isostearyl alcohol, lauryl alcohol, linoleyl alcohol, linseed alcohol, myristyl alcohol, olein alcohol, olive pomace alcohol, oleyl alcohol, olive alcohol, palm alcohol, palm kernel alcohol, palmitoyl alcohol, petroselinic alcohol, rapeseed alcohol, ricinoleyl alcohol, safflower alcohol, soy alcohol, stearyl alcohol, sunflower alcohol, tall oil alcohol, tallow alcohol, tridecyl alcohol, or technical grade mixtures thereof such as cetostearyl alcohol.

Suitable fatty alcohol ethers according to the present invention are ethers formed from the reaction of a fatty alcohol as defined above with an alkylene oxide, generally ethlyene oxide or propylene oxide. Fatty alcohols ethers can be advantageously selected from the group consisting of ceteth-20, isosteareth-10, myreth-10, laureth-16, oleth-16, polyoxyl 6 cetostearyl ether, polyoxyl 20 catostearyl ether, polyoxyl 25 cetostearyl ether, polyoxyl 2 cetyl ether, polyoxyl 10 cetyl ether, polyoxyl 20 cetyl ether, polyoxyl 4 lauryl ether, polyoxyl 9 lauryl ether, polyoxyl 23 lauryl ether, polyoxyl 2 oleyl ether, polyoxyl 10 oleyl ether, polyoxyl 20 oleyl ether, polyoxyl 2 stearyl ether, polyoxyl 10 stearyl ether, polyoxyl 21 stearyl ether or polyoxyl 100 stearyl ether.

Suitable fatty alcohol esters are the product of the reaction of a fatty alcohol as defined above and a linear of branched, saturated or unsaturated C1-C5 carboxylic acid. Examples of fatty alcohol esters are lauryl acetate, myristyl acetate, cetyl acetate, stearyl acetate and stearyl propionate.

Suitable aromatic alcohols according to the present invention can be selected from (i) arylalkanols, (ii) aryloxyalkanols (glycol monoaryl ethers) and (iii) oligoalkanol aryl ethers. The (i) arylalkanols used according to the invention have the formula Ar-(CHR)n-OH where R independently represents H or C1-C6 alkyl, with n being an integer, for example, between 1 to 10, or, for example 1 to 6, or 1, 2, 3 or 4. The group Ar can be a substituted or unsubstituted aryl group, for example phenyl or naphthyl. Example arylalkanols are benzyl alcohol, 3-phenylpropan-1-ol, phenethyl alcohol, veratryl alcohol (3,4-dimethoxyphenylmethyl alcohol) and 2-methyl-1-phenyl-2-propanol. The (ii) aryloxyalkanols used according to the invention have the formula Ar-O-(CHR)n-OH where R independently represents H or C1-C6 alkyl, with n being an integer, for example, between 2 to 10, or 2 to 6, or 2 or 3. The group Ar can be a substituted or unsubstituted aryl group, for example phenyl or naphthyl. Example arlyoxyalkanols used according to the invention are phenoxyethanol, 1-phenoxypropan-2-ol, 2-phenoxylpropan-1-ol, 3-phenoxypropan-1-ol, or mixtures thereof. The (iii) oligoalkanol aryl ethers include, for example, phenoxy diethanol, triethanol and oligoethanol, and phenoxy dipropanol, tripropanol and oligopropanol.

Suitable polyols according to the present invention include water-soluble polyols such as polyhydric alcohols with two or more hydroxyl groups in their molecule. Specific examples can include ethylene glycol, propylene glycol, 1,3-butylene glycol, 1,4-butylene glycol, hexylene glycol, dipropylene glycol, glucose, fructose, galactose, mannose, ribose, erythrose, maltose, maltitose, maltotriose, sucrose, xylitol, sorbitol, threitol, erythritol, glycerol, polyglycerol and starch alcohols. Exemplary polyols are ethylene glycol, propylene glycol, 1,3-butylene glycol, 1,4-butylene glycol, dipropylene glycol, hexylene glycol, glycerol, polyglycerol, and mixtures thereof.

Suitable alkyleneglycol esters are esters of ethylene glycol or propylene glycol with the C6-C24 fatty acids defined above. Alkyleneglycol esters can be advantageously selected from the group consisting of ethylene glycol monopalmitostearate, ethylene glycol monostearate, propylene glycol monocaprylate, propylene glycol diceprylate, propylene glycol dicaprylocaprate, propylene glycol monopalmitostearate, propylene glycol monostearate or propylene glycol alginate.

Suitable alkylene glycol ethers are polymers of ethylene oxide (polyethylene glycol monomethyl ether) or propylene oxide (polypropylene glycol monomethyl ether). Alkyleneglycol ethers can be advantageously selected from the group consisting of PEG 200, PEG 400, PEG 540, PEG 600, PEG 900, PEG 1000, PEG 1450, PEG 1540, PEG 2000, PEG 3000, PEG 3350, PEG 4000, PEG 4600, PEG 8000, PPG-9, PPG-10, PPG-17, PPG-20, PPG-26, PPG-30 or PPG-55.

As used herein, a “diluent” refers to an ingredient in a composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a composition to provide, without limitation, bulk, consistency, stability, binding ability, lubrication, disintegrating ability etc., to the composition. A “diluent” is a type of excipient.

The compositions and medicaments described herein can be administered to a human patient per se, or in compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The compositions and medicaments disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include Dexibuprofen formulated in a compatible pharmaceutical carrier may also be prepared, placed in an appropriate container, and labeled for treatment of an indicated condition.

The foregoing embodiments are merely exemplary and are not to be construed as limiting the present invention. The detailed description of the present invention is intended to be illustrative, and not to limit the scope of the claims. Many alternatives, modifications, and variations will be apparent to those skilled in the art.

### EXAMPLE 1

### In Vitro Studies

Individuals with homozygous mutations in the gene TECPR2 develop symptoms beginning in infancy. The symptoms include hypotonia, developmental delay and intellectual disability, dysmorphic facial features, spasticity and ataxia. Life expectancy is unknown due to the recent identification of this disease. However, the TECPR2 disease could be lethal in childhood.

By connecting human disease and chemical perturbation genomic data, Disease Cancelling Technology (DCT) offers an unprecedented opportunity to inform the development of new medicines. DCT identifies drug candidates by screening and identifying a single disease gene expression signature and comparing that signature to thousands of drug induced gene expression signatures.

A gene expression signature of TECPR2 disease from human patients does not exist. In order to create one, the Broad Institute's genetic perturbations gene expression database was utilized. Out of the roughly 5,000 total genetic perturbations found from mutations of the TECPR2 gene, eight genes were selected for being binding partners of TECPR2 or related to autophagosome formation (Table 1).

TECPR2 or related to autophagosome formation (Table 1)

TABLE 1
Genetic perturbations utilized for meta-analysis
of genetic perturbation gene expression data

| Gene Symbol | Gene Description |
|---|---|
| ATG12 | autophagy related 12 |
| ATG16L1 | autophagy related 16 like 1 |
| ATG3 | autophagy related 3 |
| ATG4A | autophagy related 4A cysteine peptidase |
| ATG4D | autophagy related 4D cysteine peptidase |
| ATG7 | autophagy related 7 |
| ATG 8 | GABA type A receptor associated protein like 1 |
| (GABARAPL1) | |
| NUDCD3 | NudC domain containing 3 |

ATG8 (GABARAPL1) has been described as the most important interactor for TECPR2. ATG4, ATG7 and ATG3 modify or activate ATG8. ATG8, ATG212, ATG16L1 and ATG5 form the pre-autophagosomal structure (PAS). ATG8 is also involved in COPII vesicle formation. Thus, we looked for drugs whose expression would affect these eight genes.

The dataset of drug induced gene expression signatures from the Broad institute was processed through a standard quality control procedure and served as the drug signature input to the DCT. DCT was run comparing each of the above eight genetic perturbations against the drug induced gene expression database separately to determine which drugs in the database had expression signatures that would affect each of the eight genetic perturbations derived from TECPR2 mutations. The results were meta-analyzed to create a disease cancelling score, which functions as an approximation for TECPR2 disease. Disease cancelling score is calculated by the angle between two high dimensional gene expression vectors.

### TECPR2 Results

Drugs were ranked by disease cancellation score, with a more negative score being more significant and thus more likely to affect the eight genetic perturbations found by expression of the TECPR2 gene. Dexibuprofen was a top hit, with a cancellation score of −0.94 and a meta-adjusted pvalue of 7.39E-152 (Table 2).

TABLE 2
Dexibuprofen is a top ranking drug with a strong cancellation
score and very significant meta-adjusted p value

| Compound ID | Drug name | Cancellation score | meta-Adj pval |
|---|---|---|---|
| 317909 | Dexibuprofen | −0.094 | 7.39E−152 |

Given the strong statistical significance of the dexibuprofen finding, all other NSAIDs in the database were evaluated and among them dexibuprofen remained strongest, with superiority to ibuprofen (Table 3). In addition to being the highest ranked NSAID, dexibuprofen also has a superior safety profile to acetaminophen.

TABLE 3
All NSAIDs with gene expression data were
studied, compared against all drugs tested

| preferred_name | rank | percent_rank |
|---|---|---|
| dexibuprofen | 27 | 0.06 |
| acetaminophen | 203 | 0.49 |
| piroxicam | 580 | 1.4 |
| diclofenac | 924 | 2.23 |
| ibuprofen | 1104 | 2.66 |
| nabumetone | 2244 | 5.41 |
| ketorolac | 3594 | 8.67 |
| indomethacin | 3623 | 8.74 |
| celecoxib | 3903 | 9.41 |
| salsalate | 4542 | 10.96 |
| oxaprozin | 4934 | 11.9 |
| ketoprofen | 9492 | 22.9 |
| naproxen | 10732 | 25.89 |
| sulindac | 12194 | 29.42 |
| etodolac | 13013 | 31.39 |
| tolmetin | 14248 | 34.37 |

Drugs were ranked by their disease cancellation score for TECPR2, with a smaller number in the “rank” column being a more superior drug that is likely to affect expression of the genes related to TECPR2. Dexibuprofen was the most highly ranked with a score of 27.

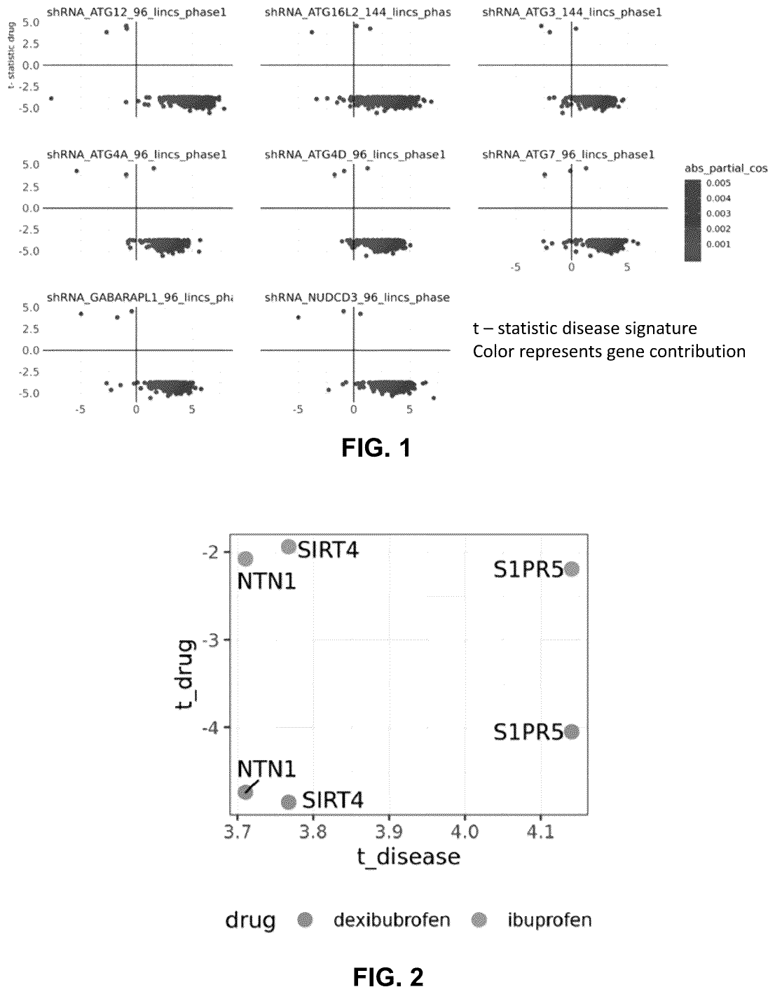

Upon investigation of the specific genes changed by dexibuprofen and in our gene expression signature of TECPR2 disease, it was observed that the majority of genes are changed in expression in the opposite direction (FIG. 1). Shown in FIG. 1 is a series of plots for the different genes responsible for the dexibuprofen high disease cancelling score for TECPR2. For example, there are individual plots for expression changes found for the ATG12, ATG16L2, ATG3, ATG4A, ATG4D, ATG7, ATG8 (GABARAPL1), and NUDCD3 genes. Each dot represents a gene. Changes in gene expression caused by dexibuprofen (y-axis) are plotted as a function of gene expression changes associated with each genetic perturbation known from TECPR2 represented (x-axis). Most genes across different genetic perturbations are located in the top left and bottom right quadrant which indicates strong reversal of the expression of genes related to TECPR2 (disease cancelling). The disease cancelling scores for each genetic perturbation were meta-analyzed to create a global TECPR2 disease cancelling score. Briefly, the DCT scores and p-values were meta-analyzed using functions from the meta R package and custom functions to combine significance values using Fisher's method. Overall, the gene expression signature of TECPR2 disease shows increased expression of many genes, and dexibuprofen works to suppress expression of these genes. Accordingly, dexibuprofen is a strong candidate treatment of individuals suffering from TECPR2 related diseases.

Genes with strong partial cosine scores were selected as “driver” genes. Out of the selected genes, S1PR5, NTN1 and SIRT4 are of high interest due to their biological relevance, (FIG. 2). Shown in FIG. 2 is a comparison of expression of three key genes between ibuprofen and dexibuprofen. Drug induced gene expression level changes is on the y-axis as t-statistic, and gene expression level changes in disease is on the x-axis as t-statistic. These three driver genes are regulated in a different direction between gene expression signatures of TECPR2 disease and dexibuprofen (orange dots), and but are merely trending with no statistically significant p-value for ibuprofen (blue dots).

Dexibuprofen more effectively suppresses expression of these three genes compared to ibuprofen. These three genes appear to be highly relevant to TECPR2 disease because they are increased in the representation of TECPR2 disease and are relevant to TECPR2 biology. NTN1 inhibits autophagy by binding the DCC receptor (Sica, V. et al. Organelle-Specific Initiation of Autophagy. Mol. Cell 59, 522-539 (2015)). S1PR5 is expressed in oligodendrocytes and we discovered that it is also modulated by dexibuprofen treatment in our data. Relevantly, oligodendrocytes are needed for maintaining the corpus callosum, which is thinned in TECPR2 disease (Covone, A. E. et al. WES in a family trio suggests involvement of TECPR2 in a complex form of progressive motor neuron disease. Clin. Genet. 90, 182-185 (2016)). Lastly, SIRT4 negatively regulates autophagy through suppression of AMPK (Han, Y., Zhou, S., Coetzee, S. & Chen, A. SIRT4 and Its Roles in Energy and Redox Metabolism in Health, Disease and During Exercise. Front. Physiol. 10, 1006 (2019); Zhao, Y. et al, RACK1 Promotes Autophagy by Enhancing the Atg14L-Beclin 1-Vps34-Vps15 Complex For ration upon Phosphorylation by AMPK. Cell Rep. 13, 1407-1417 (2015)). Other receptors and pathways represented in the list of driver genes are still under investigation.

Others have previously described the relationship between dexibuprofen (or ibuprofen) and autophagy (Peng, J. et al. Effect of Ibuprofen on Autophagy of Astrocytes During Pentylenetetrazol-Induced Epilepsy and its Significance: An Experimental Study. Neurochem. Res. 44, 2566-2576 (2019); Ikegaki, N. et al. S(+)-ibuprofen destabilizes MYC/MYCN and AKT, increases p53 expression, and induces unfolded protein response and favorable phenotype in neuroblastoma cell lines. Int. J. Oncol. 44, 35-43 (2014)) as well as myelination (Xing, B. et al. RhoA-inhibiting NSAIDs promote axonal myelination after spinal cord injury. Exp. Neurol. 231, 247-260 (2011)). The data from the findings described herein for the first time link these findings to potential therapeutic benefit in TECPR2 disease. These previous findings described RhoA inhibition as being the key driver. However, we discovered how this happens via dexibuprofen driving NTN1 expression down, and NTN1 is described to activate RhoA (Shimizu, A. et al. Netrin-1 Promotes Glioblastoma Cell Invasiveness and Angiogenesis by Multiple Pathways Involving Activation of RhoA, Cathepsin B and CREB. J. Biol. Chem. 288, (2012)).

Accordingly, embodiments of the invention related to the use of dexibuprofen as a safe treatment for TECPR2 diseases, including for pediatric use and as enhancing myelination and autophagy.

### EXAMPLE 2

### Treatment of a Patient With Hereditary Spastic Paraparesis

A human patient is tested and found to be homozygous for TECPR2 mutations and suffering from neurodegenerative issues related to Hereditary Spastic Paraparesis. The patient is given daily oral doses of dexibuprofen to reduce the effects of the disease. After 30 days of treatment, the patient is found to have reduced neurodegenerative issues relating to the TECTPR2 mutations.

What is claimed is:

1. A method of treating an Autophagy disease in a patient in need thereof, comprising administering to the patient a composition comprising a therapeutically effective amount of dexibuprofen or a pharmaceutically acceptable salt thereof.

2. The method of claim 1, wherein the Autophagy disease is an Autophagy disease related to a TECPR2 mutation in the patient.

3. The method of claim 2, wherein the method comprises treating Hereditary Spastic Paraparesis in the patient.

4. The method of claim 1, wherein the composition further comprises a pharmaceutically acceptable carrier.

5. The method of claim 1, further comprising administering another therapeutic agent to the patient.

6. The method of claim 5, wherein the composition and the other therapeutic agent are co-administered.

7. The method of claim 5, wherein both the composition and the other therapeutic agent are dispersed or dissolved together in a pharmaceutically-acceptable carrier.

8. The method of claim 1, wherein the administration is an oral administration.

9. The method of claim 8, wherein the oral administration is by a tablet comprising the composition.

10. The method of claim 1, wherein the administration is intravenous or subcutaneous administration.

11. The method of claim 10, wherein the administration is of a solution comprising dexibuprofen or pharmaceutically acceptable salt in a concentration between 1 mg/ml and 500 mg/ml.

12. The method of claim 1, wherein dexibuprofen or pharmaceutically acceptable salt thereof administered to the patient is in an amount from about 0.1 mg to about 1,000 mg of dexibuprofen per kg of body weight of the patient.

13. The use of dexibuprofen or a pharmaceutically acceptable salt thereof in the manufacture of a medicament for the therapeutic treatment of an Autophagy disease in a patient in need thereof.

14. The use of dexibuprofen or a pharmaceutically acceptable salt thereof according to claim 13, wherein the Autophagy disease is an Autophagy disease related to a TECPR2 mutation in the patient.

15. The use of dexibuprofen or a pharmaceutically acceptable salt thereof according to claim 14, wherein the method comprises treating Hereditary Spastic Paraparesis in the patient.

16. The use of dexibuprofen or a pharmaceutically acceptable salt thereof according to claim 13, wherein the medicament further comprises a pharmaceutically acceptable carrier.

17. The use of dexibuprofen or a pharmaceutically acceptable salt thereof according to claim 13, wherein the medicament further comprises another therapeutic agent.

18. The use of dexibuprofen or a pharmaceutically acceptable salt thereof according to claim 13, wherein the medicament is intended for oral administration.

19. The use of dexibuprofen or a pharmaceutically acceptable salt thereof according to claim 18, wherein the medicament is a tablet.

20. The use of dexibuprofen or a pharmaceutically acceptable salt thereof according to claim 13, wherein the medicament is intended for intravenous or subcutaneous administration.

21. The use of dexibuprofen or a pharmaceutically acceptable salt thereof according to claim 20, wherein the medicament comprises a solution containing dexibuprofen or pharmaceutically acceptable salt in a concentration between 1 mg/ml and 500 mg/ml.

22. The use of dexibuprofen or a pharmaceutically acceptable salt thereof according to claim 13, wherein medicament contains dexibuprofen or pharmaceutically acceptable salt thereof in an amount from about 0.1 mg to about 1,000 mg of dexibuprofen per kg of body weight of the patient.

23. The use of dexibuprofen or a pharmaceutically acceptable salt thereof substantially as described in the specification.
